(19) United States
(12) Patent Application Publication
(10) Pub. No.: US 2006/0089704 A1
(43) Pub. Date: Apr. 27, 2006

(54) Vascular graft and deployment system

(76) Inventor: Myles Douglas, Gardnerville, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/972,936

(22) Filed: Oct. 25, 2004

(51) Int. Cl. A61F 2/06

(52) U.S. Cl. 623/1.12; 623/1.35

(57) ABSTRACT

A vascular graft includes a main portion and a branch portion that is coupled to the main portion by an articulating joint. The vascular graft may be inserted into the thoracic aorta with the branch portion positioned within a branch vessel and the main portion positioned within the thoracic aorta. The graft may be deployed within a deployment apparatus comprising an outer member and an inner member. The outer member may include an area of increased flexibility that corresponds to the articulating joint.

### BACKGROUND OF THE INVENTION

1. Field of the Invention

The present invention relates to vascular grafts and vascular graft deployment systems.

2. Description of the Related Art

The aorta is the largest artery in the body and is responsible for delivering blood from the heart to the organs of the body. The aorta includes the thoracic aorta, which arises from the left ventricle of the heart, passes upward, bends over and passes down towards the thorax, and the abdominal aorta which passes through the thorax and through the abdomen to about the level of the fourth lumbar vertebra, where it divides into the two common iliac arteries. The thoracic aorta is divided into the (i) ascending aorta, which arises from the left ventricle of the heart, (ii) the aorta arch, which arches from the ascending aorta and (iii) the descending aorta which descends from the aorta arch towards the abdominal aortic.

A thoracic aortic aneurysm (“TAA”) is a widening, bulge, or ballooning out of a portion of the thoracic aorta, usually at a weak spot in the aortic wall. If left untreated, the aneurysm may progressively expand until the vessel dissects or ruptures. This may lead to severe and even fatal hemorrhaging. Factors leading to thoracic aorta aneurysms include hardening of the arteries (artherosclerosis), hypertension, congenital disorders such as Marfan's syndrome, trauma, or less commonly syphilis. Thoracic aorta aneurysms occur in the ascending aorta about 25% of the time, the aortic arch about 25% of the time and in the descending aorta about 50% of the time.

Treatment of thoracic aorta aneurysms depend upon the location of the aneurysm. For aneurysms in the ascending aorta or aortic arch, surgery is typically required to replace the aorta with an artificial vessel. This surgical procedure typically requires exposure of the aorta and the use of a heart-lung machine. If the aortic arch is involved, a specialized technique called “circulatory arrest” (i.e., a period without blood circulation while on life support) may be necessary. For aneurysms in the descending aorta, the vessel may also be replaced with an artificial vessel through surgery. In some circumstances, an endoluminal vascular graft may be used eliminating the need for open surgery.

As compared to, for example, the abdominal aorta artery, the thoracic aorta is a particularly difficult environment for endovascular grafts. For example, the anatomy and physiology of the thoracic aorta is more complicated than the abdominal aorta. High pulse volumes and challenging pressure dynamics further complicate endovascular procedures. Accordingly, endovascular grafts and surgery are used to treat thoracic aorta aneurysms by only the most experienced and skilled surgeons.

Accordingly, there is a general need for a endovascular graft and deployment systems for treating thoracic aorta aneurysms.

### SUMMARY OF THE INVENTION

As such, one embodiment of the present invention comprises a method of treating a thoracic aorta. The method comprises providing a vascular graft comprising a main portion and a branch portion that is coupled to the main portion, the main portion comprising a distal end and a proximal end and a main lumen extending therethrough. A catheter is provided having a distal end and a proximal end. The vascular graft is positioned within the catheter in a first, compressed state such that the branch portion is positioned closer to the distal end of the catheter than the main portion. The distal end of the catheter is advanced up through the descending aorta into a branch vessel of the thoracic aorta. The branch portion of the vascular graft is deployed within the branch vessel and then the main portion of the vascular graft is deployed in the thoracic aorta.

Another embodiment of the present invention comprises a vascular graft having a branch body with a distal end and a proximal end. The graft also includes a main body, having a distal end, proximal end and main lumen extending therethrough. An articulated joint couples the branch body to the main body such that the proximal end of the branch body generally faces the distal end of the main body. The articulated joint is configured to allow angular adjustment of the branch body with respect to the main body generally about a vertex, the vertex being moveable along a first path.

Another embodiment of the present invention comprises the combination of a deployment apparatus and a vascular graft having a main portion and a branch portion that is connected to the main portion by an articulating joint. The combination includes an elongate flexible body having a proximal end, a distal end and a region of increased flexibility located between the distal end and the proximal end. A pusher is moveably positioned within the elongate flexible body. The vascular graft is positioned within the elongated flexible body in a compressed state between the distal end of the elongate flexible body and the pusher, the vascular graft being positioned within the elongate flexible body such that the articulating joint is generally positioned within the area of increased flexibility.

Another embodiment of the present invention comprises a catheter for delivering an endovascular device to the thoracic aorta. The catheter comprises an elongate, flexible body, having a proximal end and a distal end. An endovascular device zone is positioned on the catheter for carrying a deployable endovascular device. A flex point on the catheter is positioned within the endovascular device zone. The flex point has a greater flexibility than the elongate flexible body.

Another embodiment of the present invention comprises a method of treating the thoracic aortic artery. The method comprises deploying an anchor in a branch vessel in communication with the thoracic aorta and deploying an endovascular device within the thoracic aorta. The anchor is flexibly connected to the endovascular device.

Another embodiment of the present invention comprises a method of treating a thoracic aorta, which comprises the ascending aorta, the aorta arch and the descending aorta. The method comprises providing a vascular graft comprising a main portion and a branch portion that is coupled to the main portion, the main portion comprising a distal end and a proximal end and a main lumen extending therethrough, providing a catheter having a distal end and a proximal end, the main portion of the vascular graft being positioned within the catheter in a first, compressed state and providing a removable sheath that is coupled to a pull wire for constraining the branch portion in a compressed state. The distal end of the catheter is advanced up through the descending aorta into the ascending aorta. The constrained branch portion and removable sheath are positioned at least partially within a branch vessel. The main portion of the vascular graft is positioned within the descending aorta by proximally retracting a portion of the deployment catheter. The branch portion of the vascular graft is deployed by proximally withdrawing the pull wire and removing the removable sheath from the branch portion.

Another embodiment of the present invention comprises a combination of a deployment apparatus and a vascular graft having a main portion and a branch portion that is connected to the main portion by an articulating joint. An elongated flexible body comprises an outer sheath and an intermediate member moveably positioned with the outer sheath. A removable sheath is positioned around the branch portion to constrain the branch portion in a reduced profile configuration. The main portion of the vascular graft is positioned within the intermediate member flexible body in a compressed state. The articulating joint extends through an opening in the intermediate member such that the branch portion is positioned within the elongate body between the outer sheath and the intermediate member.

Further features and advantages of the present invention will become apparent to those of ordinary skill in the art in view of the detailed description of preferred embodiments which follow, when considered together with the attached drawings and claims.

### BRIEF DESCRIPTION OF THE DRAWINGS

FIG. 1 is a schematic representation of the thoracic aorta and its principle branches.

FIG. 2a is a top plan view of the vascular prosthesis of FIG. 1a in a straightened configuration.

FIG. 2B is a side plan view of the vascular prosthesis of FIG. 1a in a straightened configuration.

FIG. 2c are front and review perspective views of a main body of the vascular prosthesis of FIG. 1a.

FIG. 2d are front and review perspective views of a branch body of the vascular prosthesis of FIG. 1a.

FIG. 3a is a side plan view of the vascular prosthesis of FIG. 1a showing the range of angular adjustment.

FIG. 3b is a side plan view of the vascular prosthesis of FIG. 1a with the with main portion rotated 180 degrees with respect to FIG. 3a and showing the range of angular adjustment.

FIG. 3c is a top plan view of the vascular prosthesis of FIG. 1a showing the range of angular adjustment.

FIG. 4 is a partial cross-sectional view of a deployment apparatus having certain features and advantages according to an embodiment of the present invention.

FIG. 4a is a closer view of a distal portion of FIG. 4.

FIG. 5 is a front view of the deployment apparatus of FIG. 4.

FIG. 6 is a schematic representation of a guidewire and deployment apparatus positioned across an aneurysm positioned in the descending aorta.

FIG. 7 is a schematic representation as in FIG. 6 with an outer sheath of the deployment apparatus proximally retracted.

FIG. 8 is a schematic representation as in FIG. 7 with the distal end of the deployment apparatus advanced into the subclavian artery.

FIG. 9 is a schematic representation as in FIG. 8 with the prosthesis deployed in the subclavian artery and the descending aorta.

FIG. 10 is a schematic representation of an aneurysm in the descending thoracic aorta with a prosthesis having certain features and advantages according to the present invention positioned therein.

FIG. 11 is a schematic representation of an aneurysm in the aortic arch of the thoracic aorta with a prosthesis having certain features and advantages according to the present invention positioned therein.

FIG. 12 is a schematic representation of an aneurysm in the ascending thoracic aorta with a prosthesis having certain features and advantages according to the present invention positioned therein.

FIG. 13 is a side view of another embodiment of a vascular prosthesis.

FIG. 14 is a front view of the prosthesis of FIG. 13.

FIG. 15 is a side view of another embodiment of a vascular prosthesis.

FIG. 16 is a front view of the prosthesis of FIG. 15.

FIG. 17a is a side view of another embodiment of a deployment apparatus comprising an outer sheath, an intermediate member and an inner core.

FIG. 17b is a side view of the deployment device of FIG. 17a with the outer sheath proximally retracted.

FIG. 17c is a side view of the distal end of the intermediate member.

FIG. 17d is a cross-sectional side view of the proximal end of the deployment device of FIG. 17a.

FIG. 18 is a schematic representation of a guidewire and deployment apparatus positioned across an aneurysm positioned in the ascending aorta.

FIG. 19 is a schematic representation as in FIG. 18 the deployment apparatus positioned across the aneurysm.

FIG. 20 is a schematic representation as in FIG. 19 with the outer sheath of the of the deployment apparatus retracted and a branch portion of the prosthesis positioned within the innominate artery.

FIG. 21 is a schematic representation as in FIG. 20 with a main portion of the prosthesis deployed in the ascending aorta.

FIG. 22 is a schematic representation as in FIG. 21 with a branch portion of prosthesis deployed within the innominate artery

### DETAILED DESCRIPTION OF THE PREFERRED EMBODIMENT

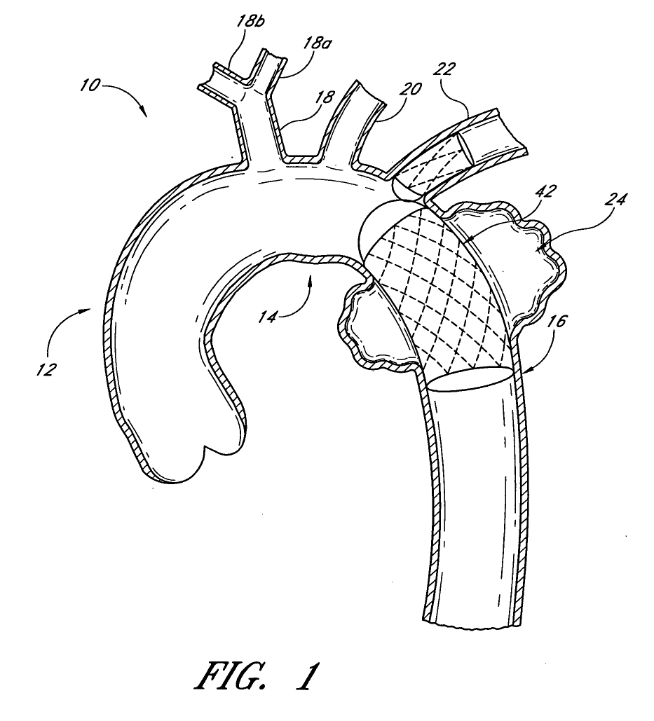

FIG. 1 illustrates a schematic representation of the thoracic aorta 10. The thoracic aorta 10 is divided into the (i) ascending aorta 12, which arises from the left ventricle of the heart, (ii) the aortic arch 14, which arches from the ascending aorta 12 and (iii) the descending aorta 16 which descends from the aortic arch 14 towards the abdominal aorta. Also shown are the principal branches of the thoracic aorta 10, which include the innomate artery 18 that immediately divides into the right carotid artery 18A and the right subclavian artery 18B, the left carotid 20 and the subclavian artery 22. An aneurysm 24 is illustrated in the descending aorta 16, just below the subclavian artery 22.

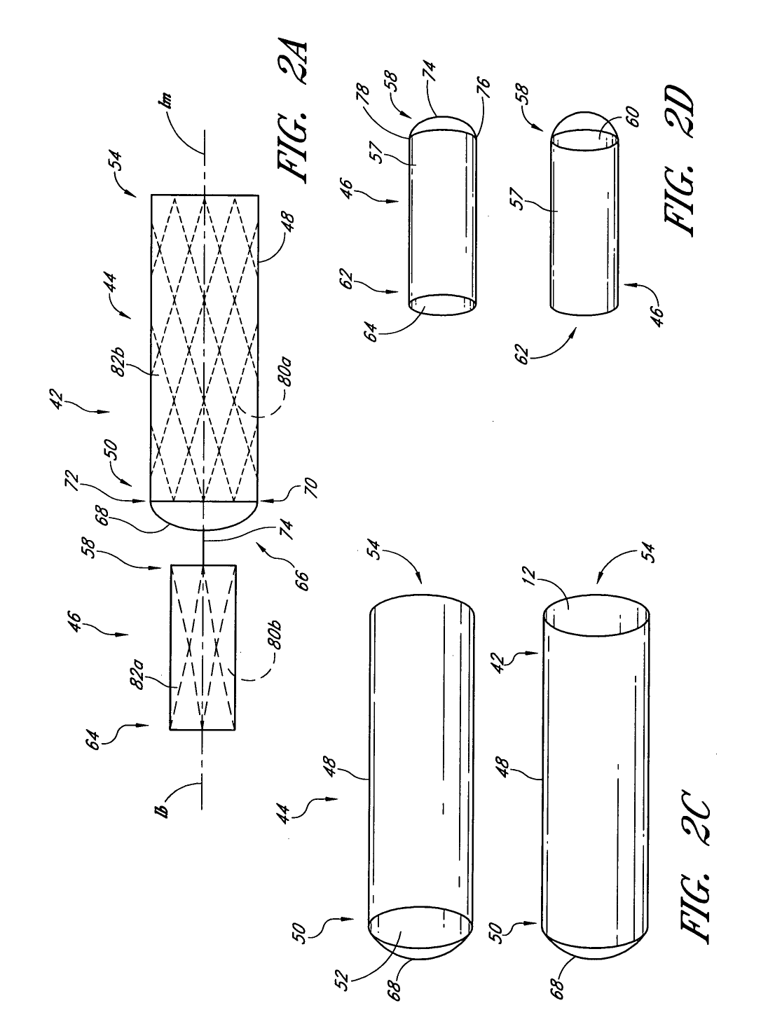

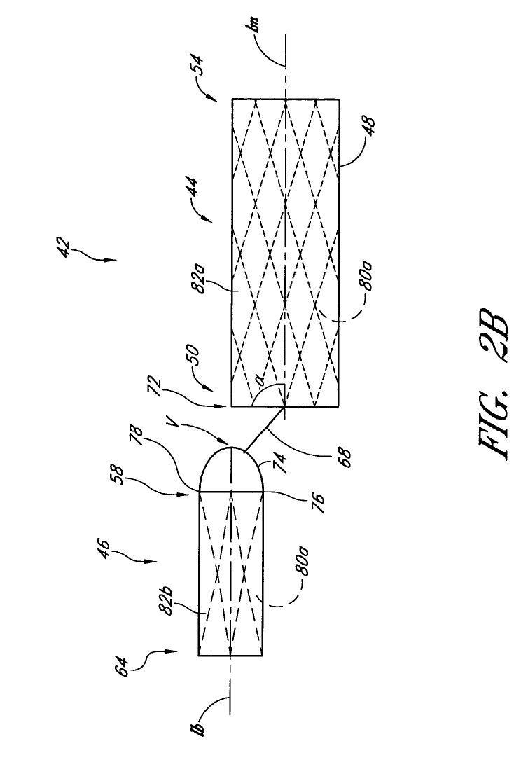

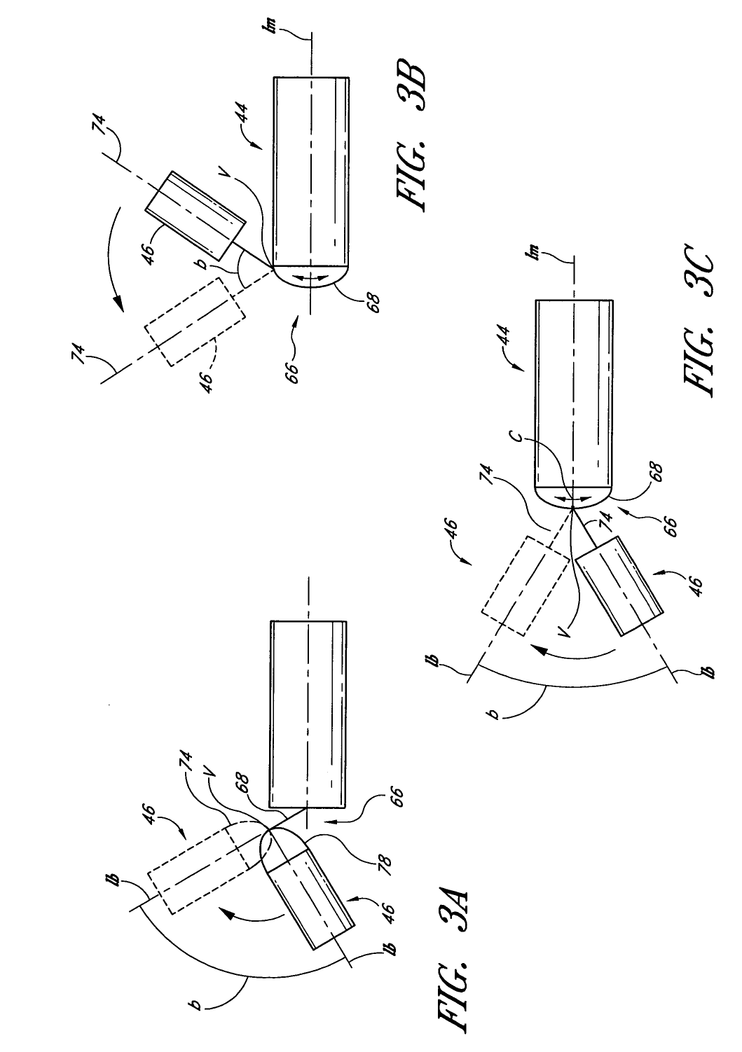

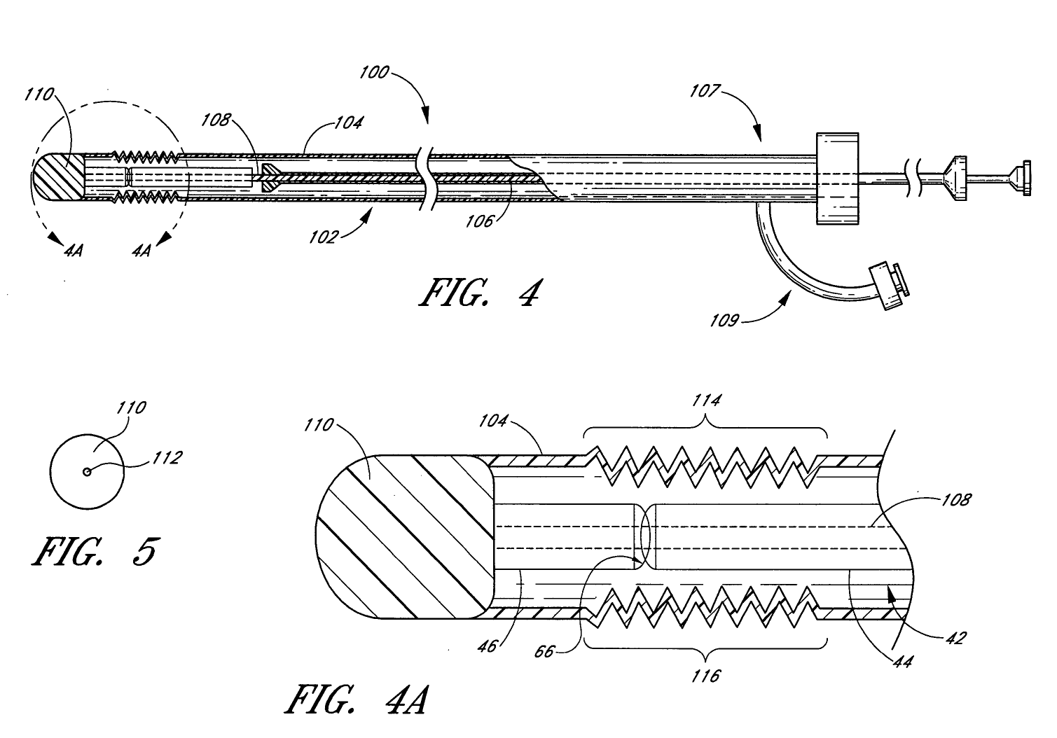

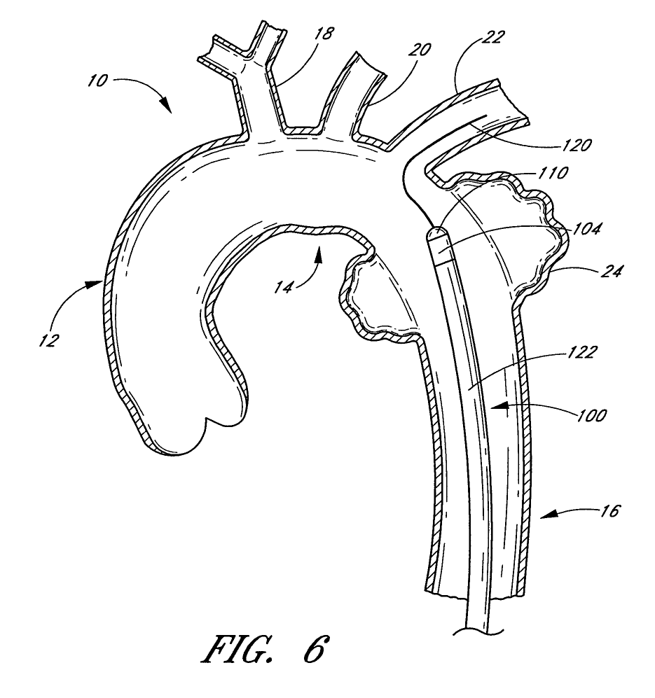

FIGS. 2A-3B illustrate an endoluminal vascular prosthesis 42, in accordance with an embodiment of the present invention. As will be explained, in more detail below, the prosthesis 42 may be used to span the aneurysm 24 as shown in FIG. 1.

With initial reference to FIGS. 2A-D, the prosthesis 42 comprises a first or main body 44 and a second or branch body 46. In the illustrated embodiment, the main body 44 comprises a generally tubular body 48 having a distal end 50, which defines a distal opening 52, and a proximal end 54, which defines a proximal opening 56 (see FIG. 2C). As used herein, the terms proximal and distal are defined relative to the deployment catheter, such that the device distal end is positioned in the artery closer to the heart than the device proximal end.

In a similar manner (see FIG. 2D), the branch body 46 comprises a generally tubular body 57 having a proximal end 58, which defines a proximal opening 60, and a distal end 62, which defines a distal opening 64. As will be explained in more detail below, in one embodiment, the main body 44 is configured such that it can extend across at least a portion of the aneurysm 24 while the branch body 46 is configured to be positioned within the subclavian artery 22.

The distal end 50 of the main body 44 and the proximal end 58 of the branch body 46 are coupled together by an articulating joint 66. In one embodiment, the articulating joint 66 is configured to axially couple the branch member 46 to the main body 46 while permitting sufficient flexibility between these bodies 44, 46 such that the branch body 46 may be placed within one of the branch vessels (i.e. the innomate artery 18, the left carotid 20 or subclavian artery 22) while the main body 44 is positioned within the thoracic aorta 10.

With reference to FIGS. 2A and 2B, in the illustrated embodiment, the articulating joint 66 comprises a first semi-circular hoop 68 having a first end 70 and a second end 72 that are coupled to the distal end 50 of the first body 44. A second semi-circular hoop 74 is provided on the branch body 46 and also has a first end 76 and a second end 78 that are attached to the proximal end 58 of the branch body 46. As shown in FIGS. 2A and 2B, the hoops 68, 74 are linked together to form the articulating joint 66. In the illustrated arrangement, the ends 76, 78 of the second hoop 74 are coupled to the proximal end 58 of the branch body 46 such that the second hoop 74 extends generally parallel to the longitudinal axis lb of the branch body 46. In contrast, the ends 70, 72 of the first hoop 68 may be coupled to the distal end 50 of the main body 44 such that the first hoop 68 forms an angle a with respect to the longitudinal axis lm of the main body 44. In this manner, as shown in FIG. 2B, the longitudinal axis lb of the branch body 46 may lie generally above or offset from the longitudinal axis lm of the main body 44. The first and second hoops 68, 74 may be attached to the main and branch bodies 44, 46 in any of a variety of ways. For example, the hoops 68, 74 may be coupled or formed as part of the tubular skeleton described below and/or coupled and/or formed with the sleeve described below.

Preferably, the articulating joint 66 provides a substantial range of motion between the main body 44 and the branch body 46. In this manner, the prosthesis 42 may be installed in a wide variety of patients in which the angles between the innomate artery 18, the left carotid 20, subclavian artery 22 and the thoracic aorta 10 may vary substantially from patient to patient. With reference to FIG. 3A which is a side elevational view of the prosthesis 42, the joint 66 preferably allows the branch body 46 to be adjusted to any of a variety of angular orientations with respect to the main body 44. The angle b represents the angular adjustment between the longitudinal axes lm, lb of the two bodies 44, 46 in a first plane generally about a vertex v positioned generally between the apexes of the first and second loops 68, 74. The angle b is limited primarily by the interference between the distal end 50 of the main body 44 and the proximal end 58 of branch body 46, and the configuration of the joint 66. It should be appreciated that the maximum angle of adjustment between the longitudinal axes lm, lb of the main and branch bodies 44, 46 in an symmetrical joint 66 as illustrated is generally half of the angle b. Depending upon the environment of use, the angle b is preferably at least about 120 degrees and often at least about 180 degrees.

With reference now to FIGS. 3B and 3C, the branch body 46 preferably includes another degree of motion with respect to the main body 44. Specifically, as shown in FIG. 3B, the vertex v about which the branch body 46 may be angularly adjusted may be moved laterally with respect to the longitudinal axis of the main body 44 as the second hoop 74 slides along the first hoop 68. This provides the articulating joint 66 with an additional range of movement and flexibility. Advantageously, with reference to FIG. 3B, this arrangement allows the main body 44 to be rotated about its longitudinal axis lm with respect to the branch body 46 while preserving at least some if not all of the angular adjustment about the vertex v described above.

In addition, or in the alternative, the articulating joint 66 may also include additional ranges of motion. For example, as shown in FIG. 3C, the illustrated embodiment advantageously allows the branch body 46 to be adjusted to any of a variety of angular orientations defined within a cone having vertex v that is generally positioned between the apexes of the first and second hoops 68, 74. The angle c represents the angular adjustment between the two bodies and the angle b is the lateral range of angular adjustment in a single plane within which the hoop 68 resides. The maximum angular adjustment between the longitudinal axes lm, lb of the main and branch bodies 44, 46 in the illustrated configuration is generally half of the angle c. Depending upon the environment of use, the angle c is preferably at least about 120 degrees and often at least about 180 degrees.

It should be appreciated that the illustrated articulating joint 66 represents only one possible configuration for the articulating joint 66 and of a variety of other articulating joint structures may be used to provide one or more of the degrees and ranges of angular adjustment described above. Such articulating joint structures include, but are not limited to mechanical linkages (e.g., inter-engaging hoops of different configurations and shapes, sliding structures, rails, hinges, ball joints, etc.), flexible materials (e.g., flexible wires, fabric, sutures, etc.) and the like.

For example, a woven or braided multi-strand connector can extend between the main body 44 and the branch body 46, without the use of first and second interlocking sliding components as illustrated. Filaments for multi-strand or single strand connectors may comprise any of a variety of metals (e.g. Nitinol, stainless steel) or polymers (e.g. Nylon, ePTFE, PET, various densities of polyethylene, etc.) depending upon the desired tensile strength and performance under continuous repeated movement. A single strand or multi-strand connector may extend from one of the main body 44 and branch body 46, with an eye on the free end, slideably carried by a hoop or strut on the other of the main body 44 and branch body 46. As a further alternative, a proximal extension of the frame work for the branch body 46 may be provided, to interlock with a distal extension of the framework for the main body 44. The use of a particular articulating joint 66 will be governed by a variety of considerations, including the desired angles of adjustability and degrees of freedom, as well as materials choices and deployment considerations which can be optimized for specific vascular graft designs.

As compared to the illustrated embodiment, such structures may be configured to have more or less range of motion and/or degrees of adjustment. For example, in some embodiments, it may be advantageous to provide angular adjustment about a vertex v between the main and branch bodies 44, 46 only within a single plane. In other embodiments, it may be advantageous to provide angular adjustment about a vertex v between the main and branch bodies 44, 46 only within a single plane while also permitting the vertex v to move about a path as described above with reference to FIGS. 3B and 3C.

With reference back to FIGS. 2A and 2B, the vascular prosthesis 42 can be formed using a variety of known techniques. For example, in one embodiment, one or both of the bodies 44, 46 comprises an expandable tubular support or skeleton 80a, 80b, and a polymeric or fabric sleeve 82a, 82b that is situated concentrically outside and/or inside of the tubular support 80a, 80b. The sleeve 82a, 82b may be attached to the tubular support 80a, 80b by any of a variety of techniques, including laser bonding, adhesives, clips, sutures, dipping or spraying or others, depending upon, e.g., the composition of the sleeve 82a, 82b and overall prosthesis design. In another embodiment, the tubular support 80a, 80b, may be embedded within a polymeric matrix which makes up the sleeve 82a, 82b.

The sleeve 82a, 82b may be formed from any of a variety of synthetic polymeric materials, or combinations thereof, including ePTFE, PE, PET, Urethane, Dacron, nylon, polyester or woven textiles. In one embodiment, the material of sleeve 82a, 82b is sufficiently porous to permit ingrowth of endothelial cells, thereby providing more secure anchorage of the prosthesis and potentially reducing flow resistance, sheer forces, and leakage of blood around the prosthesis. The porosity characteristics of the polymeric sleeve may be either homogeneous throughout the axial length of the main and branch bodies 44, 46, or may vary according to the axial position along these components. For example, with reference to FIG. 1A, it may be advantageous to configure the distal end 50 and the proximal end 54 of the main body 44, which seat against the native vessel wall, on either side of the aneurysm 24, to encourage endothelial growth, or, to permit endothelial growth to infiltrate portions of the prosthesis in order to enhance anchoring and minimize leakage. Because anchoring may be less of an issue, the central portion of the main body 44, which spans the aneurysm 24, may be configured to maximize lumen diameter and minimizing blood flow through the prosthesis wall and therefore may either be generally nonporous, or provided with pores of relatively lower porosity.

In modified embodiments, the prosthesis 42 may be provided with any of a variety of tissue anchoring structures, such as, for example, barbs, hooks, struts, protrusions, and/or exposed portions of the tubular support 80a, 80b. In other embodiments, the tubular support 80a, 80b may extend beyond one or more of the ends of the sleeve material. Such anchoring structures over time, may become embedded in cell growth on the interior surface of the vessel wall. These configurations may help resist migration of the prosthesis 42 within the vessel and reduce leakage around the ends of the prosthesis 42. The specific number, arrangement and/or structure of such anchoring structures can be optimized through routine experimentation.

In one particular embodiment, the branch body 46 comprises an uncovered stent. That is, the branch body 46 may include a tubular wire support structure 80b but does not include a sleeve, or only a portion of the branch body 46 includes a sleeve. In contrast, the main body 44, which may be used to span and isolate the aneurysm 24, is covered partly or wholly by a sleeve. In this manner, the tubular structure 80b of the branch body 46 serves to resist migration and act as an anchoring structure for the main body 44 within the thoracic aorta 10.

In still another embodiment, the branch body 46 may be used to occlude or partially occlude one of the branch vessels (e.g., the right and left carotids 18, 20 and the subclavian 22 artery). In such an embodiment, the branch body 46 may include an occluding body (not shown), such as an end cap or membrane carried by the wire support structure, which is configured to extend across the branch vessel to partially or totally occlude the vessel.

Those of skill in the art will recognize that any of a variety of tubular supports may be utilized with the illustrated embodiment. In one embodiment, the tubular supports are configured to be expanded via an internal expanding device (e.g., a balloon). See e.g., U.S. Pat. No. 6,123,722, which is hereby incorporated by reference herein. In another embodiment, the tubular support is wholly or partially self expandable. For example, a self expandable tubular support may be formed from a shape memory alloy that can be deformed from an original, heat-stable configuration to a second heat-unstable configuration. See e.g., U.S. Pat. No. 6,051,020, which is hereby incorporated by reference herein. The supports may be formed from a piece of metal tubing that is laser cut.

In another embodiment, the support comprises one or more wires, such as the tubular wire supports disclosed in U.S. Pat. Nos. 5,683,448, 5,716,365, 6,051,020, 6,187,036, which are hereby incorporated by reference herein, and other self-expandable configurations known to those of skill in the art. Self expandable tubular structures may conveniently be formed with a series of axially adjacent segments. Each segment generally comprises a zig-zag wire frame having a plurality of apexes at its axial ends, and wire struts extending therebetween. The opposing apexes of adjacent segments may be connected in some or all opposing apex pairs, depending upon the desired performance. In other embodiments, one or more of the individual segments may be separated from adjacent segments and retained in a spaced apart, coaxial orientation by the fabric sleeve or other graft material.

The tubular support or skeleton need not extend through the entire axial length of the branch and/or main bodies. For example, in one embodiment, only the distal and proximal ends 50, 54, 58, 62 of the main and branch bodies 44, 46 are provided with a tubular skeleton or support. In other embodiments, the prosthesis 42 is “fully supported”. That is, the tubular support extends throughout the axial length of the branch and/or main bodies 44, 46.

Suitable dimensions for the main and branch bodies 44, 46 can be readily selected taking into account the natural anatomical dimensions in the thoracic aorta 10 and its principal branches (i.e., the innomate artery 18, left carotid 20 and subclavian 22 arteries).

For example, main branch bodies 44 will have a fully expanded diameter within the range of from about 20 mm to about 50 mm, and a length within the range of from about 5 cm to about 20 cm for use in the descending aorta as illustrated in FIG. 1. Lengths outside of these ranges may be used, for example, depending upon the length of the aneurysm to be treated, the tortuosity of the aorta in the affected region and the precise location of the aneurysm. Shorter lengths may be desirable for the main body 44 when treating aneurysms in the ascending aorta or the aortic arch as will be appreciated by those of skill in the art.

Branch bodies 46 for use in the subclavian artery will generally have a length within the range of from about 10 mm to about 20 mm, and a fully expanded diameter within the range of from about 2 cm to about 10 cm. Both the main body 44 and branch body 46 will preferably have a fully expanded diameter in an unconstrained state which is larger than the inside diameter of the artery within which they are to be deployed, in order to maintain positive pressure on the arterial wall.

The minimum length for the main branch 44 will be a function of the size of the aneurysm 24. Preferably, the axial length of the main branch 44 will exceed the length of the aneurysm, such that a seating zone is formed at each end of the main branch 44 within which the main branch 44 overlaps with healthy vascular tissue beyond the proximal and distal ends of the aneurysm 24.

The minimum axial length of the branch body 46 will depend upon its configuration, and whether or not it includes anchoring structures such as barbs, high radial force, or other features or structures to resist migration. In general, the branch body 46 will be optimized to provide an anchor against migration of the main body 44, and may be varied considerably while still accomplishing the anchoring function.

The length of the joint is considered to be the distance between the expandable wire support for the branch body 46 and for the main body 44. In general, the length of the joint will be at least about 2 mm, and in some embodiments at least about 1 mm. Longer lengths may also be utilized, where desirable to correspond to the distance between the anatomically proximal end of the aneurysm and the desired branch vessel within which the anchoring body is to be placed. Joint lengths of at least about 50% of the expanded diameter of the branch body 44, and in some instances at least 100% and as much as 200% or more of the expanded diameter of the branch body 46 may be utilized, depending upon the anatomical requirements.

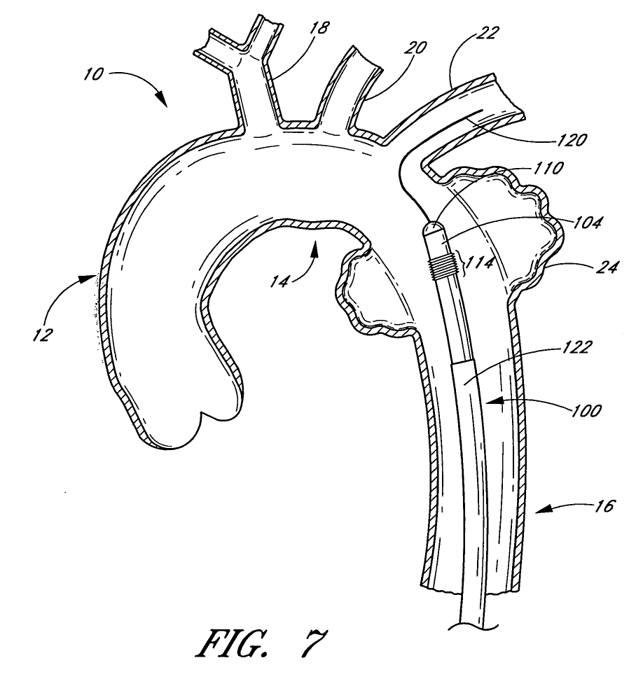

FIG. 4 is a partial cross-sectional side view of one embodiment of a deployment apparatus 100, which can be used to deploy the prosthesis 42 described above. FIG. 5 is a front view of the apparatus 100. The deployment apparatus 100 comprises an elongate flexible multi-component tubular body 102 comprising an outer sheath 104 and an inner proximal stop or pusher 106 axially movably positioned within the outer sheath 104. The outer sheath 104 may be provided with a proximal hub or valve 107 and a irrigation side arm 109, which is in fluid communication with the distal end of the catheter such as through the annular lumen formed in the space between the outer sheath 104 and pusher 106.

With continued reference to FIG. 4, a central core 108 having a smaller outer diameter than the pusher 106 may extend from the distal end of the pusher 106. A distal cap or end member 110, in turn, may be coupled to the distal end of the central core 108. A guidewire lumen 112 (FIG. 5) preferably extends through the distal cap 110, central core 108 and pusher 106.

With reference to FIG. 4A, which is a closer view of the distal end of the deployment apparatus 100, the prosthesis 42 may be positioned in a compressed or reduced diameter state within the outer sheath 104 between the distal cap 110 and the distal end of the pusher 106. As will be explained in detail below, proximal (inferior direction) retraction of the outer sheath 104 with respect to the pusher 106 will deploy the prosthesis 42

With continued reference to FIG. 4A, preferably, the outer sheath 104 includes a region of increased flexibility or articulation 114. When the prosthesis 42 is mounted within the outer sheath 104, the articulating connection 66 is preferably axially aligned with the region of increased flexibility or articulation 114. The region of increased flexibility or articulation 114 may be formed in any of a variety of manners. In the illustrated embodiment, the region of increased flexibility or articulation 114 is formed by providing the tubular member with a plurality of scores, grooves or thinned areas 116 such as a plurality of circumferential slots, which increase the flexibility of the outer sheath 104 in this region. In modified embodiments, the region of increased flexibility or articulation 114 may be formed by using a more flexible material and/or providing a mechanical linkage or a bellows configuration. In one embodiment, the central core 108 also includes an area of increased flexibility or articulation, such as an annular recess in the outer wall, which is axially aligned with the region of increased flexibility or articulation 114 on the outer sheath 104.

The tubular body 102 and the other components of the deployement apparatus 100 can be manufactured in accordance with any of a variety of techniques well known in the catheter manufacturing field. Extrusion of tubular catheter body parts from material such as Polyethylene, PEBAX, PEEK, nylon and others is well understood. Suitable materials and dimensions can be readily selected taking into account the natural anatomical dimensions in the thoracic aorta 10 and its principle branches 18, 20, 22, together with the dimensions of the desired implant and percutaneous or other access site.

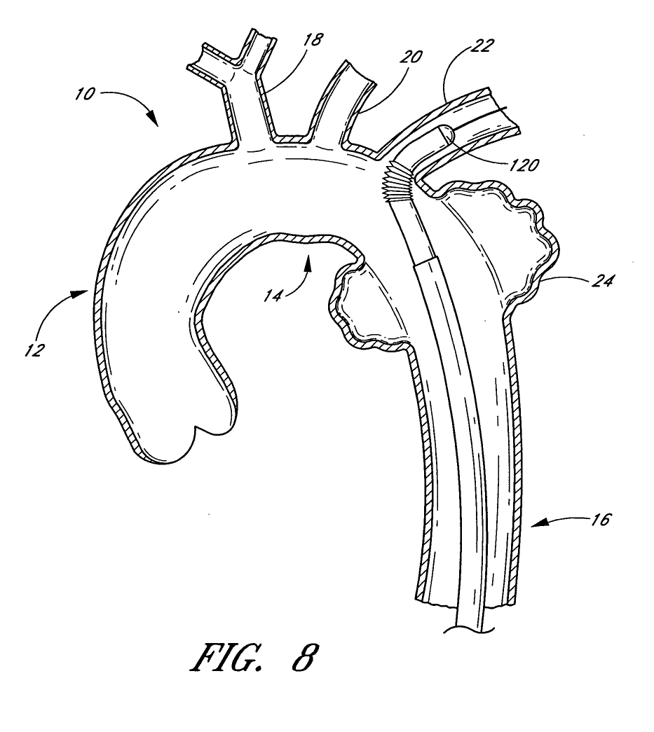

A technique for deploying the prosthesis 42 using the deployment apparatus 100 for treating an aneurysm 24 in the descending aorta 16 will now be described with reference to FIGS. 6-9. As shown in FIG. 6, a standard 0.035″ diameter guidewire 120 is preferably positioned across the aneurysm 24 and into the subclavian artery 22. The guidewire may be introduced, for example, through a percutaneous puncture, and advanced superiorly towards the aneurysm and thoracic aorta 10. In one embodiment, the percutaneous puncture is formed on the femoral artery.

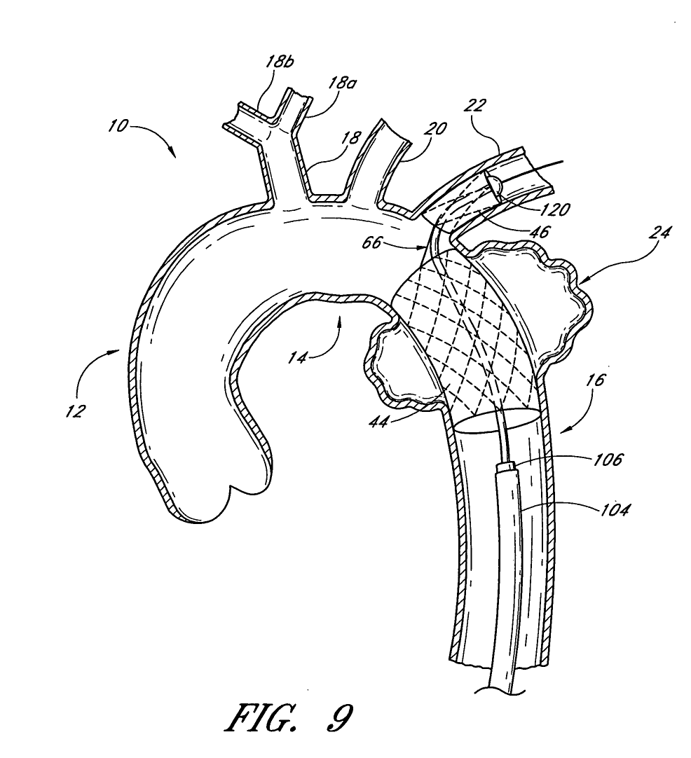

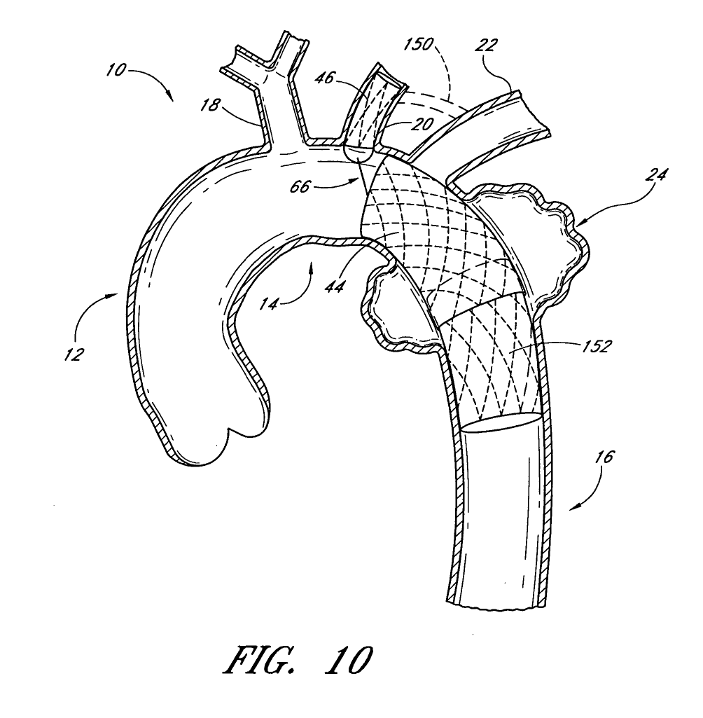

The deployment apparatus 100 is advanced over the wire until the distal end of the catheter is positioned at or near the thoracic arota. During this step, the deployment apparatus 100 may be covered at least in part by an outer tubular member 122, which preferably extends over the area of increased flexibility 114. The outer tubular member 122 advantageously increases the stiffness of the apparatus 100 thereby enhancing its pushability. As shown in FIG. 7, the outer tubular member 122 may be withdrawn exposing the area of increased flexibility 114. The distal end of the deployment apparatus may be then advanced (see FIG. 8) until the branch body (not shown in FIG. 8) within the apparatus 100 is positioned in the subclavian artery 22 and the flex point 114 is positioned in the vicinity of the ostium. The area of increased flexibility 114 advantageously facilitates advancement of the deployment apparatus 100 over the guidewire 120 and permits the catheter to navigate the tortuous turn from the descending aorta 16 into the subclavian artery 22.

With reference to FIG. 9, the outer sheath 104 may be proximally withdrawn thereby allowing the branch body 46 to expand within the branch vessel 22. Further proximal retraction, exposes the main branch 44 allowing it to expand in the thoracic aorta 10, spanning at least a portion, and more preferably the entire aneurysm 24 With the prosthesis 42 deployed, the deployment apparatus 100 may be proximally withdrawn through the deployed prosthesis 42. The deployment catheter 100 may thereafter be proximally withdrawn from the patient by way of the percutaneous access site.

The deployment apparatus 100 and/or the prosthesis 42 may include one or more radio opaque markers such that the apparatus 100 and/or the prosthesis 42 may be properly orientated with respect to the anatomy. For example, with respect to the illustrated embodiment, it is generally desirable that the first hoop 68 of the articulating joint 66 generally point towards the subclavian artery 22. Any of a variety of techniques may be used to provide radio opaque markers, such as, for example, providing the components of the deployment apparatus 100 and/or the prosthesis 42 with bands or staples made of radio opaque material or dispersing radio opaque material into the material that forms the components of the apparatus.

The illustrated embodiment has several advantages over the prior art. For example, some prior art techniques involve placing an inverted bifurcated or “Y” graft into the aorta 10 from a branch vessel. In these techniques, a deployment catheter is inserted into the aorta 10 through one of the branch vessels (typically one of the carotids 18b, 20). The legs of Y-graft are then deployed within the aorta 10 with the main trunk extending into the branch vessel. This technique has several disadvantages. For example, inserting a deployment catheter into the branch vessels, especially the carotids, may dislodge plague thereby resulting in a stroke. In addition, the deployment step may temporarily occlude the carotid areteries vessel potentially obstructing cerbaral blood flow causing severe damage to the patient. Another technique for inserting a vascular graft into the aorta 10 involves advancing a deployment catheter up through the descending aorta 16. The vascular graft is then deployed in the aorta. The vascular graft may include openings or fenestrations that must be aligned with the branch vessels. Branch grafts for the branch vessels may then be attached in situ to the main graft. Such techniques are time intensive and require a high degree skill and experience. In addition, these arrangements may create leakages near or around the fenestrations, leading to endoleaks and eventual graft failure.

In contrast, in the illustrated embodiment, the deployment apparatus 100 may be advanced through the descending aorta 16 avoiding the risks associated with advancing a catheter through the carotids. The prosthesis 42 may be deployed with the branch body 46 inserted into the branch vessel and the main body 44 in the aorta 10 by withdrawing the outer sheath 104. In this manner, the branch body 46 provides an anchor for the main body 44. This is particularly advantageous for aneurysms 24 that are positioned near a branch vessel. In such circumstances, the aorta 10 may not provide a large enough landing zone to properly support and anchor a graft positioned solely in the aorta, which may lead to endoleaks. The range of motion provided by the articulating joint 66 advantageously allows the prosthesis 42 to be used by surgeons with varying degrees of skill and experience. Specifically, because of the articulated joint 66, the prosthesis 42 may be misaligned rotationally with respect to the branch vessels.

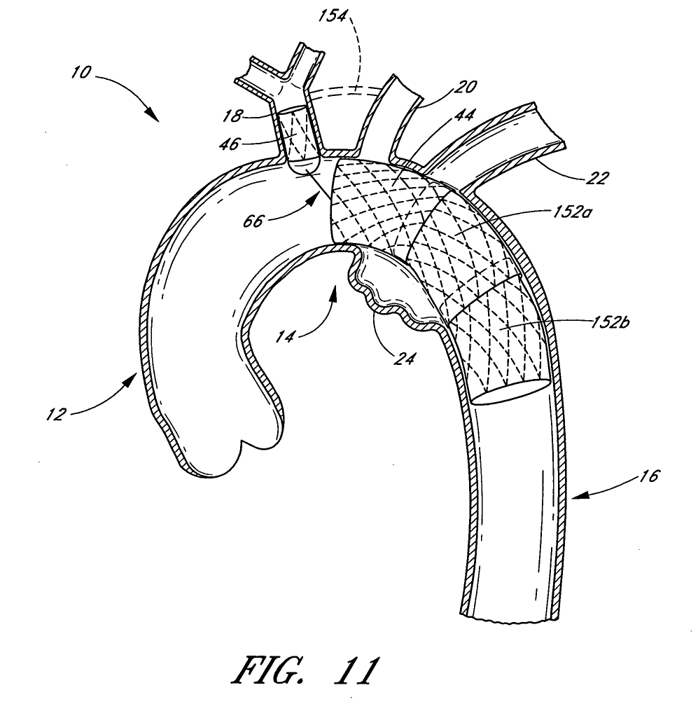

With reference to FIG. 10, the above-described procedure may be adapted to treat an aneurysm 24 positioned close the sublclavian artery 22 and/or an aneurysm that includes the subclavian artery 22. This significantly reduces the landing zone available for grafts positioned within the aorta 10. In such a procedure, the branch body 46 may be deployed within the left carotid 20 while the main body 44 may deployed at least partially within the aortic arch 14 and may extend across the subclavian artery 22. As part of such a method, a carotid-subclavian bypass 150 may be performed to direct flow from the left carotid 20 to the subclavain artery 22. In another embodiment, the main body 46 may include may include openings and/or gaps in the sleeve material to allow blood flow from the thoracic aortic artery into the subclavian artery 22. Other arrangements for allowing blood from the aorta 10 to pass through the prosthesis 42 may also be used. For example, the porosity of the sleeve in the main body 44 may be increased and/or various holes or openings may be formed in the sleeve.

As shown in FIG. 10, an extension or cuff graft 152 may be positioned within the main body 44 to effectively lengthen the prosthesis 42. In one embodiment, the cuff 152 may be arranged in a similar manner as the main body 44. The cuff 152 may be deployed with a second deployment apparatus and in a manner such that the distal end of the cuff 152 is expanded within proximal end of the main body 44 in an overlapping relationship. In some embodiments, it may be advantageous to provide any of a variety of complementary retaining structures between the main body 44 and the cuff 152. Such structures include, but are not limited to, hooks, barbs, ridges, grooves, etc. The cuff 152 may be attached in situ (see e.g., U.S. Pat. No. 6,685,736, the disclosure of which is hereby incorporated by reference in its entirety herein) or before deployment.

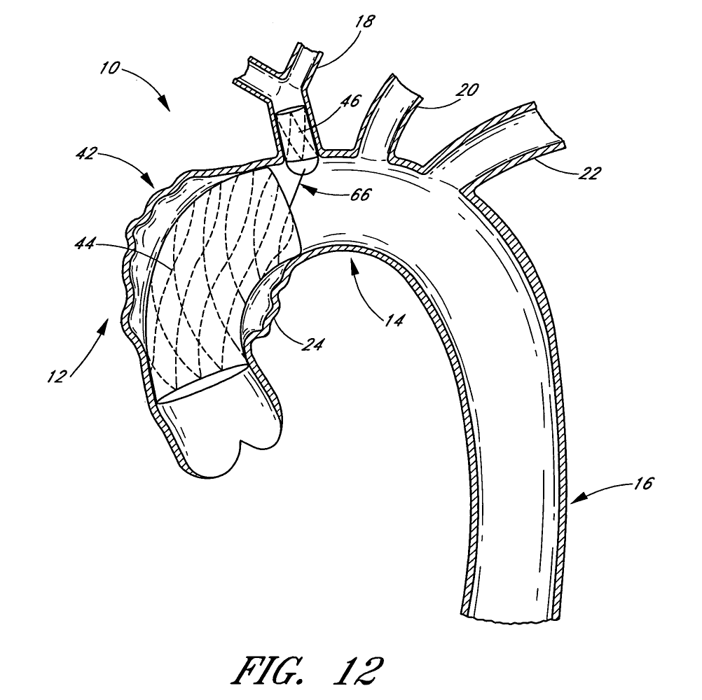

With reference to FIG. 11, the above-described procedure may also be adapted to treat an aneurysm 24 positioned in the aortic arch 14. For example, the branch body 46 may deployed in the in a manner similar to that described above. The main body 44, in turn, may extend across the left carotid 20 and/or subclavian artery 22. One or more cuffs 152a, 152b may be provided and deployed as described above, to extend the prosthesis 42 through the aortic arch 14 to isolate the aneurysm 24. In another embodiment, the main body 44 may be configured to extend through the entire aortic arch 14. As shown in FIG. 11, in embodiments where the left carotid and/or subclavian are effectively closed by the main body 44 and/or the cuffs 152a, 152b, a carotid to carotid by pass 154 may be accomplished using open surgical techniques. In a modified embodiment, the main body 44 and/or cuffs 152a, 152b may include openings and/or gaps in the sleeve material to allow blood flow into the left carotid 20 and/or subclavian artery 22. As described above, other arrangements for allowing blood to pass through the prosthesis 42 may also be used.

FIG. 12 illustrates the prosthesis 42 described above placed within the aorta 10 to isolate an aneurysm 24 in the ascending aorta 14. In this embodiment, the deployment apparatus 100 may be inserted into the aorta 12 from the innomate artery 18 and the main branch 44 may be deployed first by proximally withdrawing the outer sheath 104 into the right carotid innomate artery 18.

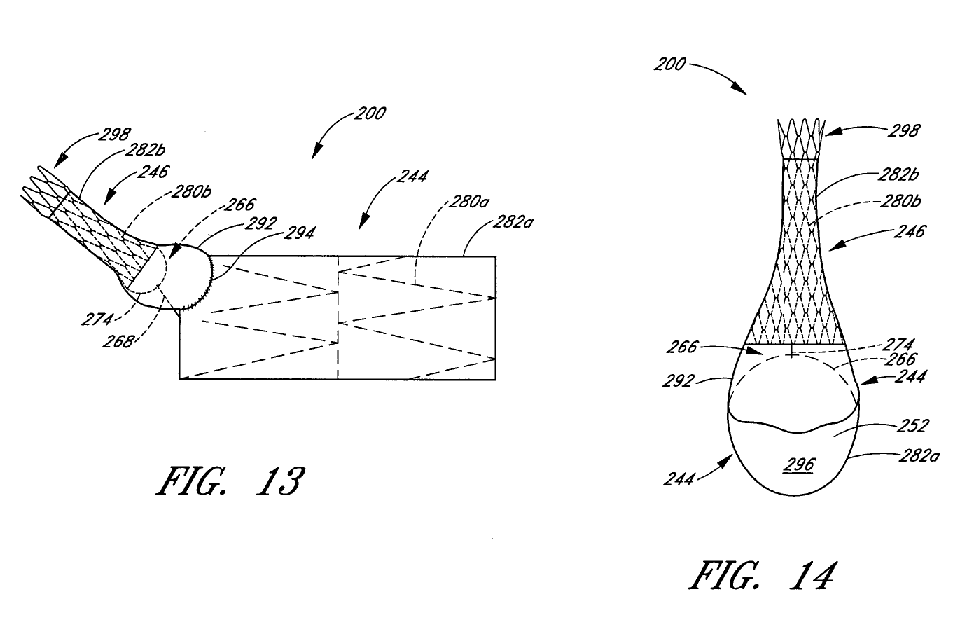

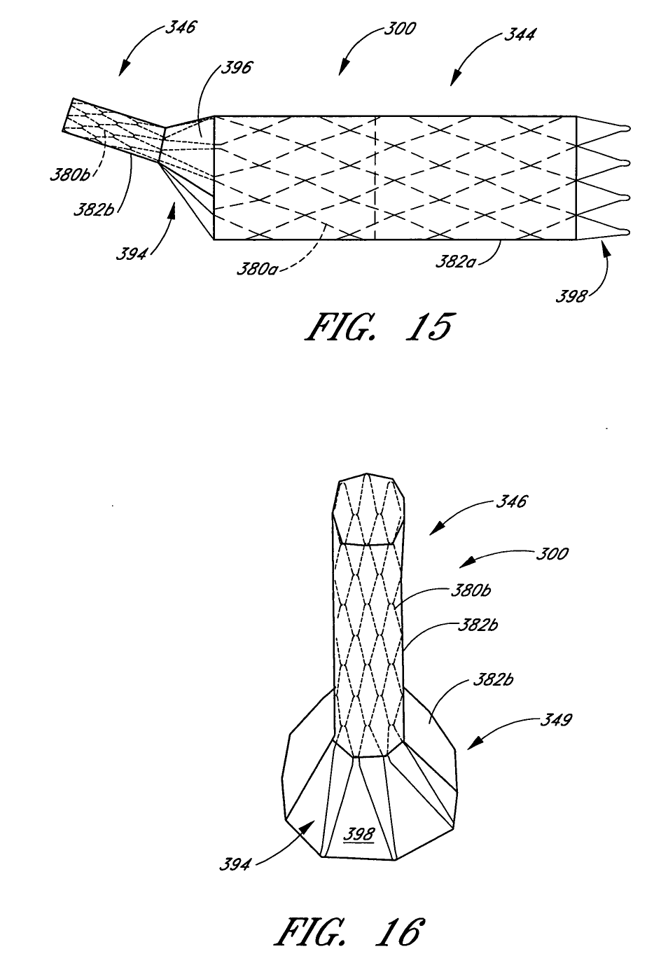

FIGS. 13 and 14 are side and front views, respectively, of a modified embodiment of vascular graft 200. In these figures, like elements to those shown in FIGS. 2A-2D are designated with like reference numerals, preceded by the numeral “2”. As shown, the vascular graft 200 generally comprises a first or main body 244 and a second or branch body 246, which are coupled together by an articulating joint 266. As described above, the articulating joint 266 may be configured as described above and in the illustrated embodiment includes a first hoop 268 and a second hoop 274. The bodies 244, 246 may comprise a tubular support or skeleton 280a, 280b and a polymeric or fabric sleeve 282a, 282b as described above.

In this embodiment, a connection portion 292 extends between the fabric sleeves 282a, 282b of the bodies 244, 246. The connection portion 292 generally extends over the articulating joint 266 and may be formed of the same material as the sleeves 282a, 282b. In the illustrated embodiment, the connection portion 292 is an extension of the sleeve 282b of the branch body 246 that is attached to the sleeve 282a of the main body 244 by stitches 294. Of course, various other configurations may be used to form the connection portion 292. The connection portion 292 is configured to leave at least a portion 296 of the distal opening 252 of the main body 244 open such that fluid may flow into the main body 244. This embodiment may be particularly advantageous for aneurysms positioned near, at and/or within a branch vessel to the thoracic aorta 10. In such applications, the connection portion 292 may extend across the aneurysm thereby isolating the aneurysm.

With continued reference to FIGS. 13 and 14, in the illustrated arrangement, a portion 298 of the tubular skeleton 280b of the branch body 246 extends distally beyond the end of the sleeve 282b to provide an additional distal anchoring mechanism for the branch body 246 as described above.

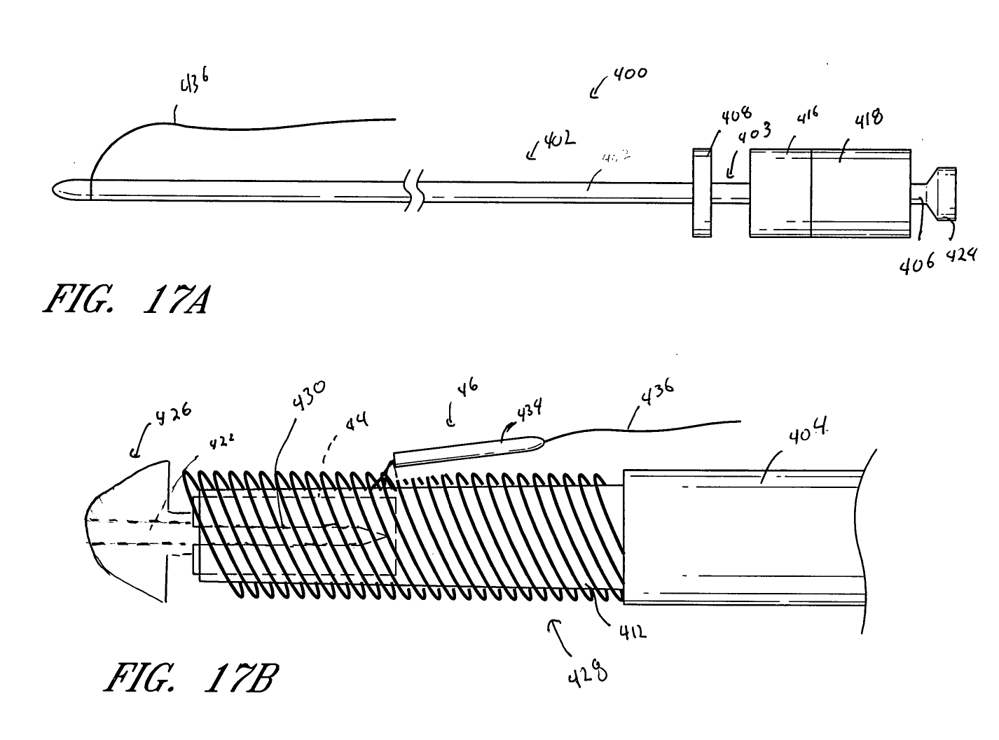

FIGS. 15 and 16 are side and front views, respectively, of another modified embodiment of vascular graft 300. In these figures, like elements to those shown in FIGS. 2A-2D are designated with like reference numerals, preceded by the numeral “3”. As with the previous embodiment, the vascular graft 300 generally comprises a first or main body 344 and a second or branch body 346, which are coupled together by an articulating joint 366. The bodies 344, 346 may comprise a tubular support or skeleton 380a, 380b and a polymeric or fabric sleeve 382a, 382b as described above.

In this embodiment, the articulating joint 366 is formed by connecting the tubular supports 380a, 380b of the main and branch bodies 344, 346. In this manner, a portion 394 of the tubular support extends between and connects the bodies 344, 346. In one embodiment, the bodies 344, 346 from a single body support or skeleton that comprise the main and branch bodies 344, 346 and the connection portion 394 extending therebetween.

The connection portion 394 is preferably be configured to allow articulation of the branch body 346 with respect to the main body 344 as described above. As with the previous embodiment, a portion 396 of the tubular sleeve may also extend between the main and branch bodies 344, 366. As shown in FIG. 16, a distal opening 398 remains in the sleeve to allow flow into the main branch 344 and exposing a portion of the connecting portion 394. As with the previous embodiment, this embodiment may be particularly advantageous for aneurysms positioned near, at and/or within a branch vessel to the thoracic aorta 10. In such applications, the connection portion 392 may extend across the aneurysm thereby isolating the aneurysm.

With continued reference to FIGS. 15 and 16, in the illustrated arrangement, a portion 398 of the tubular skeleton 380a of the main body 344 extends distally beyond the end of the sleeve 382a to provide an additional proximal anchoring mechanism for the main body 344 as described above.

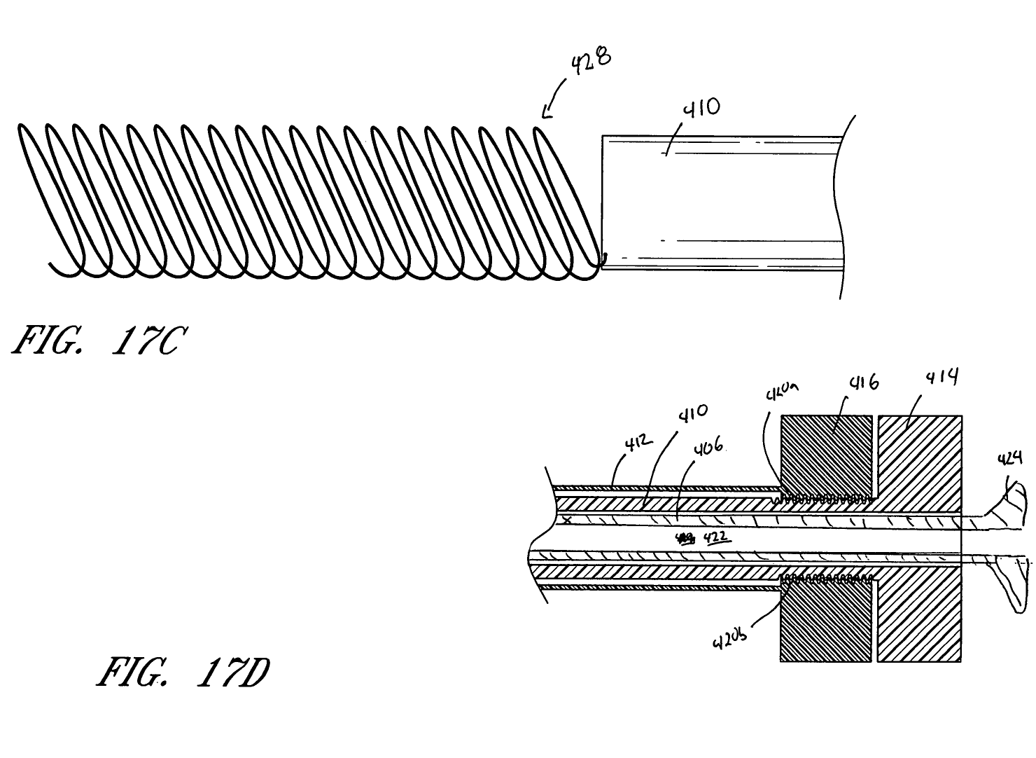

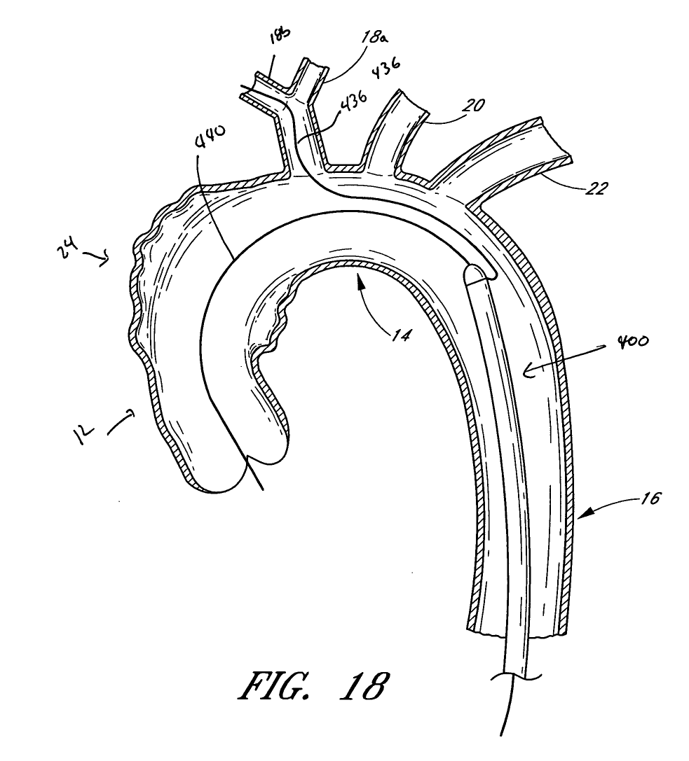

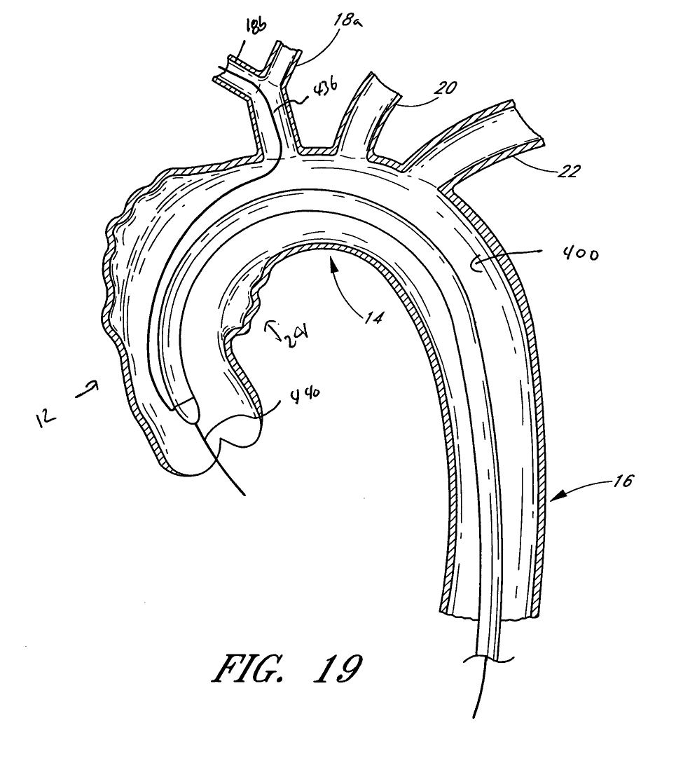

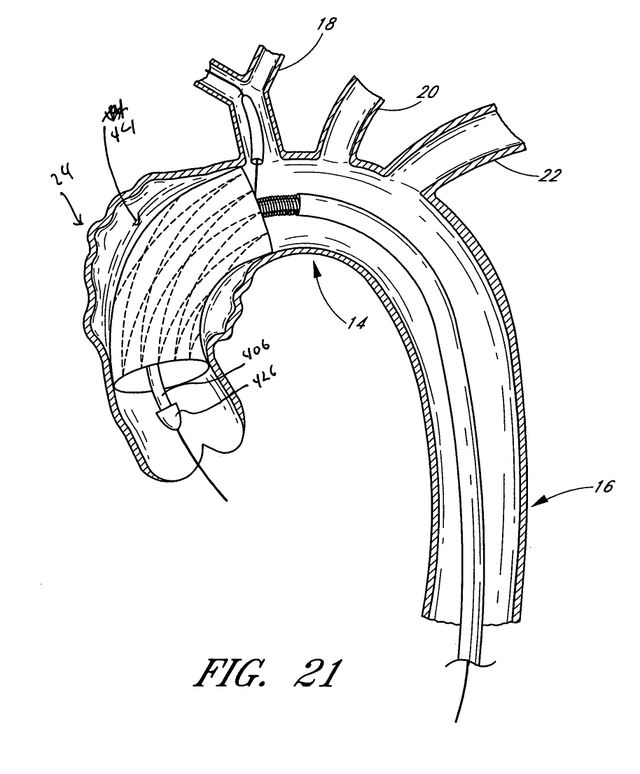

As mentioned above, with reference to FIG. 12, in certain embodiments, the prosthesis 42 described above may be used to isolate an aneurysm 24 in the ascending aorta 14. FIGS. 17A-22 illustrate one embodiment of a deployment device 400 and a method for deploying the prosthesis 42 within the ascending aorta 14.

With initial reference to FIGS. 17A-D, the deployment device 400 for placing a prosthesis in the ascending aorta 14 generally comprises an elongate flexible multi-component tubular body 402 comprising an outer sheath 404, an intermediate member 403, and an inner core 406. As will be explained below, the intermediate member 403 and the core 406 are preferably axially movablely positioned within outer sheath 402. With reference to FIG. 17A, the outer sheath 402 may be provided with a proximal hub 408.

With reference to FIGS. 17C-D, the intermediate member 403 comprises an inner member 410, which is axially and preferably also rotationally moveably positioned within an outer member 412. Both members 410, 412 extend from a distal end of the outer sheath 404 to the proximal end of the outer sheath 404 and terminate at proximal hubs 414, 416. As mentioned above, the inner member 410 is preferably able to rotate with respect to the outer member 412. Preferably, the apparatus 400 includes a mechanism for limiting and/or controlling the rotational movement between the two members 410, 412. As shown in FIG. 17D, in the illustrated embodiment, this mechanism comprises corresponding threads 420a, 420b positioned on the proximal portions of the inner member 410 and outer member 412 respectively. Of course in modified embodiments, other mechanisms may be used, such as, for example, corresponding grooves or protrusions.

As best seen in FIG. 17D, the inner core 406 extends through the inner member 410. The inner core 406 defines a guidewire lumen 422 that extend through the inner core 406 from its distal end to proximal end. The proximal end of the inner core 406 may include a hub 424. As seen in FIG. 17B, the distal end of the inner core 406 forms a nose cone or cap 426. As shown in FIG. 17A, the distal end of the outer sheath 402 may abut against the nose cone 426 to provide the deployment device 400 with a tapered or smooth distal end.

With reference now to FIG. 17C, the distal end of the inner member 410 includes a helical coil 428. The helical coil 428 may be formed from any of a variety of materials including a metallic wire. As explained below, the helical coil 428 is configured to restrain the main branch 44 in a reduced profile configuration while providing an opening through which the joint 66 between the main body 44 and branch body 46 may extend. In the illustrated embodiment, this opening is defined by the spaces between the coils of the helical coil. With reference to FIG. 17B, the distal end of the outer member 412 advantageously extend through the coil 428. In this manner, the outer member 412 lies between the main body 44 and the coil 428 and minimizes the chances that the main body 44 is snagged or entrapped by the coil 428 during deployment. In modified embodiments, the deployment apparatus 400 may be used without the outer member 412. The distal end of the outer member 412 includes one or more openings or slits 430 through which the joint 66 may extend. As explained below, the slits 430 also allow the distal end of the outer member 412 to expand as the coil 428 is retracted and the main body 44 expands to its unconstrained diameter.

FIG. 17B shows the distal end of the deployment device 400 with the outer sheath 402 retracted to expose the distal end of the inner and outer members 410, 412. As shown, the main body 44 is constrained with in the coil 428. The linkage 66 extends through the gaps 530 in the outer member 412 and between the coil 428. The branch body 46, in turn, is constrained within a tubular sheath 434. The sheath 434 is attached to a pull wire 436, which is used to remove the sheath 434 as explained below. When the outer member 404 is not retracted, the branch body 46 lies within the sheath 434 between the coil 428 and the outer sheath 404. In other embodiments, the coil 428 may be replaced with constraining member having any of a variety of slots and openings which constrain the main body 44 while providing an opening for the linkage 66 to move through as the outer member 410 is retracted to release the main body 44.

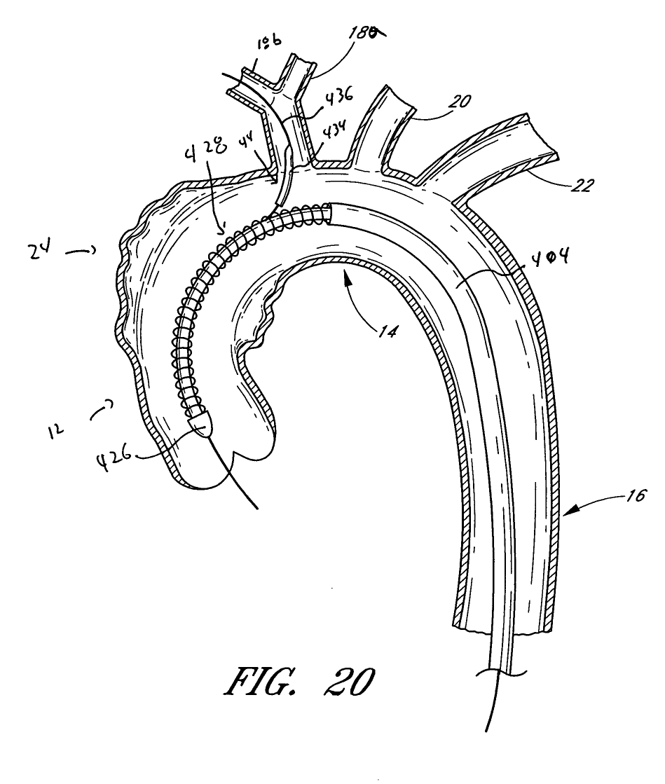

A technique for deploying the prosthesis 42 using the deployment apparatus 400 described above for treating an aneurysm 24 in the ascending aorta 12 will now be described with reference to FIGS. 18-22. In a preferred embodiment, access to the right brachial and left common femoral arteries is provided through the use of insertion sheaths (not shown) as is well know in the art. A guidewire (not shown) is inserted from the right brachial through the left femoral artery. A guiding catheter may then be inserted through the right brachial over the guidewire to the left femoral. After the guiding catheter is in place, the guidewire may be removed. A second guidewire 440 is inserted through the formal access sight and into the aorta 10 until its distal end is positioned in the ascending aorta just above the aortic valve. The pull wire 436 of the deployment apparatus may then be introduced into the guiding catheter until it emerges from the right brachial. In this manner, pull wire 435 may be positioned into the right subclavian artery 18B as shown FIG. 18. The guiding catheter may then be removed and the deployment device 400 may be advanced over the second guidewire 440 into the aorta 10 as shown in FIG. 18.

With reference to FIG. 19, the deployment device 400 is advanced over the guidewire 440 until the distal end of the device is just above the aortic valve. The outer sheath 404 is then retracted to expose the coil 428 and release the branch body 46 constrained within the sheath 435. The pull wire 436 and the apparatus 400 may be adjusted to position the branch body 46 properly within the innomate artery 18. In a modified embodiment, the outer sheath 404 is retracted before the device 400 is advanced into the descending aorta. 12.

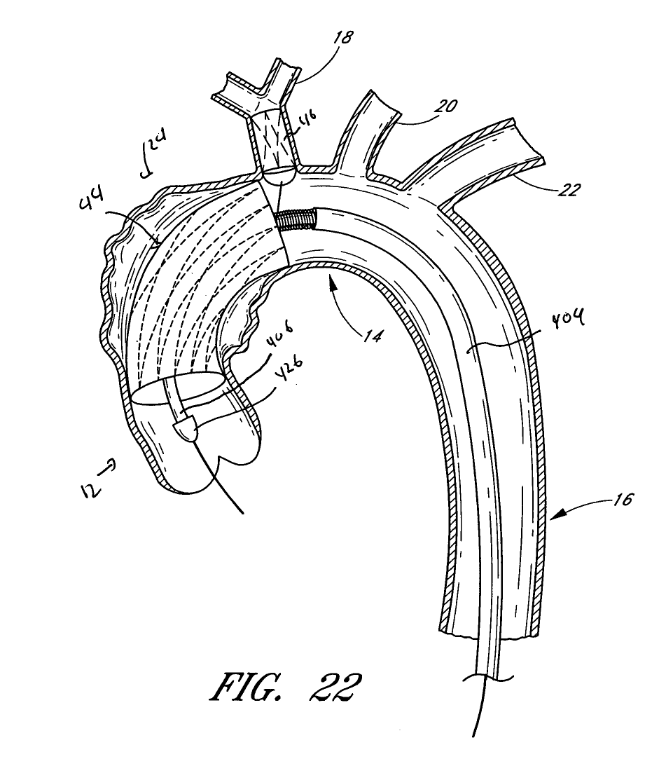

With the branch body 46 and main body 44 in the desired location, the inner member 410 is rotated with respect to the outer member 412. This causes the coil 428 to unscrew proximally as the linkage 66 moves through the spaces between the coils and the distal end of the coil 428 retracts to expose the distal end of the branch body as shown in FIG. 21. The inner member 410 is preferably rotated until the coil 428 has retracted sufficiently to fully deploy the main body 44 as shown in FIG. 21. With the main body 44 deployed, the pull wire 424 may be withdrawn to pull the sheath of the branch body 46 deploying the branch body 46 within the innomate artery 18. The distal end of the deployment apparatus 400 may then be withdrawn through the deployed prosthesis 42 and withdrawn from the patient.

In modified embodiments, several features of the above described method and apparatus for deploying the prosthesis 42 in the ascending aorta 12 may be modified. For example, one or more of the procedures described above may be omitted or rearranged. In addition, the apparatus 400 may be modified. For example, as mentioned above, the coil 428 may be replaced with a tubular member comprising slots through which the linkage 66 may extend. The tubular member may then be withdrawn while the proximal end of main branch is held in place by a pusher. In this manner, the main branch 44 may be pushed out of the tubular member to deploy the main branch body 44.

The apparatuses and methods described above have been described primarily with respect to thoracic aorta and aneurysms positioned therein. However, it should be appreciated that the apparatuses and methods may also be adapted for aneurysms and defects in other portions of the vascular anatomy. For example, it is anticipated that the apparatuses and methods described above may find utility in treating aneurysms or other defects in the abdominal aorta and/or its related branch vessels.

While a number of preferred embodiments of the invention and variations thereof have been described in detail, other modifications and methods of using and medical applications for the same will be apparent to those of skill in the art. Accordingly, it should be understood that various applications, modifications, combinations, sub-combinations and substitutions may be made of equivalents without departing from the spirit of the invention or the scope of the claims.

What is claimed is:

1. A method of treating a thoracic aorta, which comprises the ascending aorta, the aorta arch and the descending aorta, the method comprising:
providing a vascular graft comprising a main portion and a branch portion that is coupled to the main portion, the main portion comprising a distal end and a proximal end and a main lumen extending therethrough;
providing a catheter having a distal end and a proximal end, the vascular graft being positioned within the catheter in a first, compressed state such that the branch portion is positioned closer to the distal end of the catheter than the main portion;
advancing the distal end of the catheter up through the descending aorta into a branch vessel of the thoracic aorta; and
deploying the branch portion of the vascular graft in the branch vessel and then deploying the main portion of the vascular graft in the thoracic aorta.

2. The method as in claim 1, comprising providing the catheter with a tubular member and a pusher moveably positioned within the tubular member, at least a portion of the vascular graft being positioned within the tubular member between the pusher and the distal end of the catheter.

3. The method as in claim 2, wherein deploying the branch portion of the vascular graft in the branch vessel comprises proximally withdrawing the tubular member with respect to the pusher.

4. The method as in claim 3, wherein deploying the main portion of the vascular graft within the aorta comprises proximally withdrawing the tubular member further with respect to the pusher.

5. The method as in claim 1, further comprising providing a second vascular graft and deploying the second vascular graft having a distal end and a proximal end with a second lumen extending therethrough and deploying the second vascular graft within the aortic artery to place the second lumen in fluid communication with the main lumen.

6. The method as in claim 1, wherein the main portion of the vascular graft spans, at least in part, an aneurysm in the thoracic aorta.

7. The method as in claim 1, wherein the branch vessel is the innomate artery.

8. The method as in claim 1, wherein the branch vessel is the left carotid.

9. The method as in claim 1, wherein the branch vessel is the subclavian artery.

10. The method as in claim 1, further comprising inserting a by-pass between at least two of the branch vessels.

11. The method as in claim 1, wherein the main portion and the branch portion are coupled together by an articulating joint.

12. The method as in claim 1, wherein the main portion and the branch portion comprise a tubular support extending between the main portion and the branch portion.

13. A endovascular graft, comprising:
a branch body, having a distal end and a proximal end;
a main body, having a distal end, proximal end and main lumen extending therethrough; and
an articulated joint that couples the branch body to the main body such that the proximal end of the branch body generally faces the distal end of the main body, the articulated joint configured to allow angular adjustment of the branch body with respect to the main body generally about a vertex, the vertex being moveable along a first path.

14. The vascular graft as in claim 13, wherein the branch body and the main body each comprise self-expandable tubular frame.

15. The vascular graft as in claim 14, wherein the main body includes a polymeric sleeve.

16. The vascular graft as in claim 14, wherein the branch body includes a polymeric sleeve.

17. The vascular graft as in claim 13, wherein the articulated joint comprises a first loop and a second loop, the first loop having a first end and a second end coupled to the main body a distal portion extending distally from the distal end of the main body, the second loop having a first end and a second end coupled to the branch body and a proximal portion extending proximally from the proximal end of the branch body, the first and second loops being interconnected with each other.

18. The vascular graft as in claim 17, wherein the first and second loops are substantially semi-circular in shape.

19. The vascular graft as in claim 17, wherein the distal portion of the first loop extends from the distal end of the main body in a direction that is generally transverse to a longitudinal axis of the main body

20. The vascular graft as in claim 19, wherein the proximal portion of the second loop extends from the proximal end of the branch body in a direction that is generally parallel to a longitudinal axis of the branch body

21. The vascular graft as in claim 17, wherein the first path is defined, at least in part, by the distal portion of the first loop.

22. The combination of a deployment apparatus and a vascular graft having a main portion and a branch portion that is connected to the main portion by an articulating joint, comprising:
an elongated flexible body having a proximal end, a distal end and an region of increased flexibility located between the distal end and the proximal end; and
a pusher moveably positioned within the elongated flexible body;
wherein the vascular graft is positioned within the elongated flexible body in a compressed state between the distal end of the elongated flexible body and the pusher, the vascular graft being positioned within the elongated flexible body such that the articulating joint generally positioned within the region of increased flexibility.

23. A catheter for delivering an endovascular device to the thoracic aorta, comprising:
an elongate, flexible body, having a proximal end and a distal end;
an endovascular device zone on the catheter, for carrying a deployable endovascular device;
a flex point on the catheter, within the endovascular device zone, the flex point having a greater flexibility than the elongate flexible body.

24. A catheter for delivering an endovascular device to the thoracic aorta as in claim 23, comprising an inner core and an outer sleeve.

25. A catheter for delivering an endovascular device to the thoracic aorta as in claim 24, wherein the flex point comprises at least one opening in the wall of the tubular sleeve.

26. A catheter for delivering an endovascular device to the thoracic aorta as in claim 24, wherein the flex point comprises a plurality of circumferentially extending slots in the wall of the tubular sleeve.

27. A catheter for delivering an endovascular device to the thoracic aorta as in claim 23, wherein the endovascular device zone has a proximal limit and a distal limit, and the flex point is closer to the distal limit than the proximal limit.

28. A catheter as in claim 27, wherein the flex point is positioned about 10 mm to about 30 mm from the distal edge of the catheter.

29. A method of treating the thoracic aortic artery, comprising the steps of:
deploying an anchor in a branch vessel in communication with the thoracic aorta; and
deploying an endovascular device within the thoracic aorta;
wherein the anchor is flexibly connected to the endovascular device.

30. A method of treating the thoracic aortic artery as in claim 29, wherein the deploying an anchor step comprises deploying a stent in the branch vessel.

31. A method of treating the thoracic aortic artery as in claim 30, wherein the stent is a self expanding stent.

32. A method of treating the thoracic aortic artery as in claim 29, wherein the branch vessel is the subclavian artery.

33. A method of treating a thoracic aorta, which comprises the ascending aorta, the aorta arch and the descending aorta, the method comprising:
providing a vascular graft comprising a main portion and a branch portion that is coupled to the main portion, the main portion comprising a distal end and a proximal end and a main lumen extending therethrough;
providing a catheter having a distal end and a proximal end, the main portion of the vascular graft being positioned within the catheter in a first, compressed state;
providing a removable sheath that is coupled to a pull wire for constraining the branch portion in a compressed state,
advancing the distal end of the catheter up through the descending aorta into the ascending aorta;
positioning the constrained branch portion and removable sheath at least partially within a branch vessel;
deploying the main portion of the vascular graft within the descending aorta by proximally retracting a portion of the deployment catheter; and
deploying the branch portion of the vascular graft by proximally withdrawing the pull wire and removing the removable sheath from the branch portion.

34. The method as in claim 33, comprising positioning the proximal end of the pull wire through the innominate artery and through an access site in the right brachial.

35. The method as in claim 33, comprising retracting an outer sheath of the catheter to expose the constrained branch portion of the vascular graft.

36. The method as in claim 33, wherein deploying the main portion of the vascular graft comprises rotating a portion of the catheter.

37. The combination of a deployment apparatus and a vascular graft having a main portion and a branch portion that is connected to the main portion by an articulating joint, comprising:
an elongated flexible body comprising an outer sheath and an intermediate member moveably positioned with the outer sheath;
a removable sheath positioned around the branch portion to constrain the branch portion in a reduced profile configuration;
wherein the main portion of the vascular graft is positioned within the intermediate member flexible body in a compressed state, the articulating joint extending through an opening in the intermediate member such that the branch portion is positioned within the elongate body between the outer sheath and the intermediate member.
